(19) United States
(12) Patent Application Publication
(10) Pub. No.: US 2005/0265923 A1
(43) Pub. Date: Dec. 1, 2005

(54) Embolus therapy using insoluble microparticles or vesicles containing contrast agents

(76) Inventors: John Luke Toner, Libertyville, IL (US); Gerald Lee Wolf, Winchester, MA (US); Daryl Michael Simmons, Niantic, CT (US); Gregory Lyan McIntire, Westchester, PA (US); Edward Richard Bacon, Audabon, PA (US); Kathleen Illio, Phoenixville, PA (US)

Correspondence Address: AMERSHAM HEALTH, IP DEPARTMENT, 101 CARNEGIE CENTER, PRINCETON, NJ, 08540-6231, US

(21) Appl. No.: 11/165,259

(22) Filed: Jun. 23, 2005

Related U.S. Application Data
(62) Division of application No. 09/425,289, filed on Oct. 25, 1999.
Application No. 09/425,289 is a continuation of application No. PCT/GB98/01195, filed on Apr. 24, 1998.
(60) Provisional application No. 60/057,073, filed on Aug. 27, 1997.

(30) Foreign Application Priority Data
Apr. 24, 1997 (GB) 9708250.7
Nov. 26, 1997 (GB) 9725007.0

(51) Int. Cl. A61K 51/00

(52) U.S. Cl. 424/1.11; 424/9.36

(57) ABSTRACT

The invention provides a method of embolus therapy comprising administering into the vasculature of a perfused zone of tissue in a human or non-human animal subject a composition comprising particles of a size or formulation selected to generate emboli at a target site within said subject, characterised in that as said particles are used solid water-insoluble particles of a non-radioactive diagnostically effective compound or vesicles encapsulating a non-radioactive diagnostically effective compound or a solution thereof, and in that embolus location is detected by a diagnostic imaging technique.

This invention relates to improvements in and relating to methods of embolus therapy, e.g., methods for the treatment of tumors, vascular malformations and other vascular disorders where surgery may not be a viable option or for reducing bleeding during surgery, and to pharmaceutical compositions used in such methods.

While emboli cause stoppages of blood flow and are normally considered to be undesirable and sometimes are life-threatening, embolus generating agents have been used in certain fields of medical treatment, generally to block off blood supply to tumors or to tissue when the intention is to induce ischemia. In the case of tumor therapy, embolization, optionally combined with chemotherapy (chemoembolization), achieves a beneficial cytotoxic effect. In other cases, blood loss is reduced and surgery is facilitated. In either case the embolus generating agent is usually administered via a catheter into an artery upstream of the site at which embolus formation is to occur.

Because embolus formation is generally undesirable, in embolus therapy it is particularly desirable that the embolus formed should be detectable by a diagnostic imaging modality (such as X-ray, MR imaging or ultrasound). However of the embolus generating agents currently in medical practice, only Lipiodol™ (Ethiodol™) is amenable to imaging.

Lipiodol comprises an iodinated fatty acid ester derived from poppyseed oil and is observed by radiographic imaging to show where the embolus has localized. This approach however has the drawback that the oil disperses within the body as droplets which are susceptible to breaking up to form smaller droplets which may pass downstream of the target embolus site and cause emboli to form in tissues remote from the target organ, e.g., in the lungs. As a result significant adverse events can result from this misdirected migration of the oily agent. The embolus may lodge too proximally to the intended site, allowing collateralization of the target bed and may also translocate after an uncertain time. Thus with Lipiodol the behaviour of the embolic material in use cannot be accurately predicted. As pointed out by Takeda et al., Lipiodol has no anti-cancer effect and little embolic effect (Takeda et al., Adv. in X-ray Contrast, vol. 4, 30-33 (1997)).

Other conventionally used embolus generating agents, such as gelfoam, are not themselves detectable by imaging modalities and require the administration of a conventional water-soluble contrast agent (e.g., an X-ray agent such as Omnipaque or an MRI contrast agent such as Omniscan or Magnevist) to enable the location of the embolus to be determined. This may be done by tracking the blood vessel of interest to detect the point at which contrast enhancement ceases. It is assumed that the embolus is located at the point where contrast agent is blocked from further passage down the vessel of interest. This can however result in inaccurate diagnoses and diminished prognoses for the patient if the embolus is not actually located at the point where contrast enhancement stops being evident on the image.

Novel, nonaqueous solutions and suspensions have recently been reported for use as embolus causing agents Ethanol solutions of methacrylate polymers (A. Sadato et al., Acad. Radiology vol. 5(3), 198-206 (1998)) and ethylene vinyl alcohol copolymer (J. C. Chaloupka et al., Am. J. Neuroadiology vol. 15(6), 1107-1115 (1994)) have been reported to be unpredictable and exhibit technical difficulties and adverse outcomes. Dimethyl sulfoxide (DMSO) solutions of PVA with suspended particles of barium sulfate or tantalum oxide (WO 97/04656) have been reported and embody similar problems. Dimethyl sulfoxide (DMSO) solutions of a pre-polymer with suspended particles of barium sulfate or tantalum oxide (WO 97/04657) have also been reported and embody similar problems.

The use as chemoembolization agents of agarose gel particles loaded with a cytotoxic agent or a soluble X-ray contrast agent has been proposed by Kishi et al. in Nippon Acta Radiologica 55: 300-304 (1995). However the quantity of contrast agent that can be loaded into the matrix of a carrier particle is limited due to the size constraints on the overall particle (the particle size is dictated by the diameter of the blood vessel in which location of the embolus is sought—too large and the particle will not reach the desired location, too small and the particle will pass downstream of the desired location with the accompanying risk of adverse events mentioned above for Lipiodol) and the proportion of particle volume made up by the matrix material itself. Moreover detection of the embolus by imaging requires very small concentrations of embolus forming particles to be detectable and agarose gel particles of appropriate size containing X-ray contrast agents will not provide a satisfactory contrast enhancement for monitoring both initial placement of the embolus, and long term monitoring of the therapeutic application.

While radiolabels could be detected even when present at very small concentrations at an embolus, the use of radiopharmaceuticals is generally complex (e.g., requiring the generation of the radiolabel shortly before administration) and is not preferred by the medical community. Furthermore, scintigraphy is not usable for monitoring the intervention in progress, i.e., determining the targeting, dosimetry etc. Moreover the embolus generating agent may remain in place for a prolonged period and in such circumstance s the use of radiolabels is again not preferred. Finally, the embolic agent(s) and the radiolabel may become separated, thus giving false information regarding the location of the embolus itself.

There is thus a need for a non-radioactive embolus generating agent which is contrast effective at the concentrations achievable at the embolus, which is not prone to forming undesired emboli at locations remote from the target tissue, and which may be used to monitor placement and longer term persistence of the embolus.

It has now been found that solid water-insoluble particles of one or more non-radioactive diagnostically effective compounds and vesicles encapsulating one or more non-radioactive diagnostically effective compounds or a solution thereof may be used effectively as embolus generating agents and that the emboli thereby generated are detectable by diagnostic imaging modalities.

The nature of these materials is such that the solubility is less than 10 mg/ml in water and more preferably less than 1 mg/ml, while most preferably the solubility of these embolic agents is less than 100 micrograms/ml of water. Even further, the, solubility of these embolic agents will be less in plasma than measured in water due to the presence of salts and plasma-resident proteins and opsonins. Thus, the solubility of the particles envisioned in this invention is preferably less than 10 mg/ml and particularly preferably less than 1 mg/ml, and most preferably less than 100 micrograms/ml in water and even less soluble in plasma.

Thus viewed from one aspect the invention provides a method of embolus therapy comprising administering into the vasculature of a human or non-human animal (preferably mammalian) subject a composition comprising particles of a size or formulation selected to generate emboli at a target site within said subject, characterised in that as said particles are used solid water-insoluble particles of a non-radioactive diagnostically effective compound or vesicles encapsulating a non-radioactive diagnostically effective compound or a solution thereof, and in that embolus location is detected by a diagnostic imaging technique. The objective is to reduce and/or stop vascular perfusion or extravasation of the target region.

Viewed from a further aspect the invention also provides the use of solid water-insoluble particles of a non-radioactive diagnostically effective compound or vesicles encapsulating a non-radioactive diagnostically effective compound or a solution thereof for the manufacture of an embolus generating pharmaceutical composition for use in therapy.

Viewed from a yet further aspect the invention also provides the use of solid water-insoluble particles of a non-radioactive diagnostically effective compound or vesicles encapsulating a non-radioactive diagnostically effective compound or a solution thereof for the manufacture of an embolus generating pharmaceutical composition for use in chemoembolus therapy.

By diagnostically effective it is meant that the compound is capable of detection by a diagnostic imaging modality, e.g., X-ray, ultrasound, MRI, magnetotomography, light imaging (including near infra red imaging) or electrical impedance tomography, and thus that emboli created by the particles comprising such diagnostically effective compounds may be located and monitored by such imaging modalities. Such compounds will generally be referred to herein as contrast agents. By therapy, it is meant that therapeutic materials may be deposited, in accordance with the invention, in a precise location by embolization.

The particles used according to the invention are either particles of a solid contrast agent or are particles (vesicles) encapsulating a contrast agent which may be in solid, liquid or gas phase. In the former case, the particles may comprise a core surrounded by a coat and the solid contrast agent may make up either the core or, more preferably, the coat. Where the contrast agent forms the coat (e.g., about a polymer bead) it will be water-insoluble, while where it forms the core it will be water-insoluble if the coat is porous or water soluble. With a water insoluble coat a water soluble solid contrast agent core may be used.

The precise structure adopted for the particles will to a large extent depend on the means by which the contrast agent achieves contrast enhancement in the chosen imaging modality. Thus, for example for X-ray imaging techniques, contrast enhancement is generally achieved by X-ray attenuation by heavy atoms in the contrast agent. The attenuation effect is not dependent on the chemical environment of these heavy atoms (high density materials) and accordingly the contrast agent may be on the outside or on the inside of the particle or may make up the entire particle. For T₁ dependent MRI contrast agents, contrast effect is dependent on chemical environment and accordingly the contrast agent should form the surface of the particle or should be in an aqueous environment in the core of a vesicle. For such T₁ contrast agents, the particle may advantageously be a porous particle of or containing a water-insoluble or non-leaching contrast agent. Insoluble metal compounds such as gadolinium oxide and gadolinium oxalate are preferred.

For particulate T₂ or T₂* agents, e.g., superparamagnetic metal oxide crystals, the particles may conveniently be held by a polymeric carrier, e.g., of a biodegradable polymer, so that eventual biodegradation of the polymer releases the particulate contrast agent and removes the blockage to blood flow, or in certain circumstances where a more permanent blockage is required, the polymeric carrier may be refractory or the particles alone may be sufficient to cause the embolus.

For ultrasound imaging techniques, the contrast agent should be echogenic and may suitably be a gas (or a gas precursor which generates a gas at body temperatures) enclosed within a vesicle which causes embolization at the desired site.

It is also evident that the embolic particles of this invention can be combined with any conventional contrast agent of any modality to image a zone of reduced perfusion (tissue distribution). For example, any X-ray (i.e., Hypaque, Omnipaque) or MRI (i.e., Omniscan) contrast agent could be added to these embolic suspensions to image not only the embolus but the entire zone of reduced perfusion. The same could hold for any conventional ultrasound or nuclear imaging agent. The added contrast agent reflects the tissue pharmacokinetics of similarly delivered therapeutic agents to confirm the therapeutic arena in any given instance.

The contrast agent in the particles used according to the invention may for example be a water-insoluble solid iodinated organic compound, e.g., a triiodophenyl compound such as those described in U.S. Pat. No. 5,318,767, U.S. Pat. No. 5,451,393, U.S. Pat. No. 5,352,459, U.S. Pat. No. 5,569,448, e.g., NC 8883, NC 67722 or NC 12901. Other X-ray contrast agent particles may be produced by coating a particle (e.g., a glass, polymer or inorganic solid bead) with an insoluble X-ray opaque compound. This would reduce the load of contrast agent and yet provide contrast during imaging. Thus for example a suspension of polymer beads in a solution of a water-insoluble iodinated contrast agent in a non-aqueous solvent may be mixed with water to cause the iodinated agent to precipitate on the bead surface to produce a particle sufficiently large to be an embolus generator and sufficiently X-ray dense to be visualizable. Particle size may be controlled by the rate of water addition and by the amount of water added prior to particle recovery by filtration or centrifugation. Further suitable X-ray agents include water-insoluble iodinated liquids provided with a surface coating or crosslinked at the surface to prevent particle break up on administration.

Insoluble metal oxides and metal salts, (e.g., sulfides and sulfates) (e.g., of metals of atomic number greater than 22) may also be used as embolus generators. Thus for example particles of insoluble metal oxides and salts are available commercially in a range of particle sizes from 0.1 mm to 1 mm and larger. Zirconium oxides, zirconium silicates, yttrium oxides and other transition metal oxides may be mentioned in this regard and may be obtained commercially. Similarly beads of inert metals such as gold or platinum may be used in this regard. These materials are X-ray dense and very inert; moreover they can readily be purified by heat depyrogenation and steam sterilization. Much smaller metal oxide particles (e.g., titanium oxides) are also available, e.g., from vapour deposition processes. Again these can readily be purified by the same techniques. Tungsten oxides either alone or in combination with other metals are particularly suitable due to their X-ray opacity.

A particular interesting group of insoluble metal salts are the phosphate salts of formula I, as described in Narasaraju T. S. B. and Phebe D. E., “Some physico-chemical aspects of hydroxyapatite”, J. Mat. Sci. 31: 1-21 (1996), which is herein incorporated by reference, and wherein formula I can be represented by:
M₁₀(PO₄)₆A_{z}.
wherein
M=Ba, Ca, Cd, Mg, Pb or Sr
A=OH⁻, Cl⁻, F⁻ or CO₂⁻²
Z=2 if A is univalent, 1 if A is divalent.

Particularly preferred is Ca₁₀(PO₄)₆OH₂, known as hydroxyapatite.

This material is the major component of bone and is porous. It is commercially available from a number of vendors and can be processed to very small particle sizes, although particle sizes in the range of tens of microns are generally desired for the present invention. This material, either suspended in water or in the presence of conventional soluble X-ray contrast agents is X-ray dense (i.e., like bone) and causes the desired embolic effect throughout the capillary bed of exposed tissues. Examples have been reported of the use of very small particles of hydroxyapatite (<200 nm) for MRI contrast in liver, spleen and blood after doping with a magnetically active metal ion like manganese or mixed iron oxides (see U.S. Pat. No. 5,560,902, U.S. Pat. No. 5,419,892, and U.S. Pat. No. 5,342,609). Those examples are included herein by reference for the preparation of embolic particles having those same MRI activities for diagnostic MRI imaging of embolized tissues. In addition, hydroxyapatite is expected to have advantages in drug delivery over particles of pure X-ray contrast agents inasmuch as it is porous and can be used to sequester therapeutic moieties, such as oncologics and biologics such as TNF, IL1, IL2, etc., promoters and inhibitors of vascular growth, as listed below, and radioactive nuclei for interstitial radiotherapy of tumors and other lesions.

Where the contrast agent is to function as an MRI contrast agent, especially as a T₁ agent, it may be particularly advantageous to deposit the contrast agent on a particle. The particle can comprise a polymer such as polystyrene or polylactic acid, polycyanoacrylate, polymethacrylate, polylactide-co-glycolide or polyvinylalcohol, or can be a glass or ceramic particle (e.g., SiO₂, ZrO, ZrSiO₂, TiO₂, AlO₃ etc.) or in porous particle (e.g., a zeolite) of the appropriate size. By way of example, mixing a low pH solution of gadolinium (III) chloride and a high pH solution of sodium oxalate with stirring in a vessel containing the carrier particle in suspension would cause precipitation of gadolinium oxalate, an MRI active solid, on or in the carrier particle. Alternatively, the particle can be an oil droplet suspended in an injectable aqueous medium. The core may be a diagnostically useful material for the same modality as the coating, i.e., both coating and core are X-ray opaque material, or the core may be a diagnostically useful material for a different modality as the coating, i.e., the core may be MR active while the coating is an acoustically active material.

As ultrasound embolization agents one may use vesicles (e.g., liposomes, micelles or microballoons) containing an echogenic gas or gas precursor (e.g., air, oxygen, nitrogen, carbon dioxide, helium, sulphur hexafluoride, low molecular weight hydrocarbons, or fluorocarbons (e.g., perfluoroalkanes such as perfluorobutane or perfluoropentane)). The vesicle membrane may be for example a lipid (or mixture of lipids) or it may alternatively be a polymer. Where ultrasound destruction of the vesicles is desired, the membrane will preferably be relatively frangible, e.g., as in the Cavisome product of Schering AG. The ultrasound embolizing agents will preferably be coformulated with conventional (smaller and/or more flexible) echogenic ultrasound agents (e.g., gas filled vesicles) to enable embolus placement to be followed more readily. Alternatively the suspension medium for the embolization agent may contain a surfactant and may be shaken to produce surfactant-stabilized microbubbles before administration.

Simple polymer beads, or particles of a chromophore optionally provided with a light transmitting coating, can be used according to the invention as light imaging effective embolus generating agents. Likewise, for magnetotomography magnetic particles (i.e., ferro-, ferri- or superparamagnetic particles, e.g., iron oxide or mixed oxide particles) may be used as detectable embolus generating agents. In this case the particles may be composite particles of a non-magnetic matrix and one or more magnetic particles and as the matrix one will preferably use a biodegradable polymer so that on degradation the magnetic particles are released and in due course taken up by the reticuloendothelial system. In instances where a more permanent embolus is desired, the polymer may be refractory to degradation or the magnetic particles may themselves be of such a size as to form the embolus without need of a matrix polymer.

In one preferred embodiment, the embolization agent according to the invention comprises particles of polyvinylalcohol (PVA) incorporating a diagnostically effective material, e.g., a paramagnetic or superparamagnetic material, an iodinated X-ray contrast agent or a heavy metal compound, etc. as discussed herein. For this invention, these PVA particles will preferably have a particle size below 50 μm, especially below 20 μm, so as to function as capillary embolic agents. Moreover, they may advantageously be treated so that they are highly charged or are coated with a charged coating material, e.g., a surfactant. Particles incorporating paramagnetic or heavy metal ions or compounds or insoluble salts thereof or iodinated organic compounds are particularly preferred as these may be produced in a straightforward fashion. Thus such particles can be prepared by equilibrating PVA particles in a solution of the metal ion of interest (e.g., Mn, Fe, Gd, Dy, W, Ba, etc.) such that the pores and surfaces of the PVA particles act like an ion exchange resin and adsorb the metal ions of interest. This will normally be done in a low pH, aqueous solution wherein the particles swell some 20% in volume and the metal ions are soluble. After equilibration, the particles can be separated by filtration or centrifugation or any other physical method from the solution phase. This can also be done by diafiltration. The particles may then be resuspended in an elevated pH solution such that the adsorbed metal ions are converted to insoluble metal oxides thus yielding PVA particles with entrapped heavy metal particles for CT and/or MRI contrast. Alternatively, the metal ions can be precipitated with salt solutions rather than the elevated pH. For example, Mn can be precipitated by the addition of carbonate, phosphate, or silicate while Fe can be precipitated with any number of salts including analytical reagents and some iodinated contrast agents like sodium hypaque, and sodium iodipamide. Thus, PVA particles can be prepared via relatively simple solution chemistry which have either MRI or CT dense particles encapsulated within. PVA particles may likewise be produced with both MRI and CT dense agents encapsulated by using a mixture of metal ions in the initial solution equilibration.

The particle containing compositions used according to the invention will advantageously comprise a liquid (preferably aqueous) carrier medium and preferably that carrier medium will contain a dissolved or smaller particulate contrast agent, particularly preferably an agent effective for contrast enhancement in the same imaging modality as the embolus generating particles. These dissolved or smaller contrast agents may be diagnostically effective in the same or in a different imaging modality as the larger embolus generating particles. For example iohexol may be used in conjunction with an MRI-active embolic agent or gadodiamide may be used in conjunction with an X-ray opaque embolic agent. In this way the placement of the embolus may be detected even more effectively in real time. The addition of these soluble agents allows imaging of the zone of reduced perfusion as well as the embolus itself. While the extra contrast agent is preferably in solution or suspension in the carrier medium (e.g., being a soluble iodinated X-ray contrast agent such as iohexol, iodixanol, iopamidol, ioversol, iotrolan, metrazamide, etc. or a soluble MRI contrast agent such as Gd DTPA, Gd DTPA-BMA, Gd DOTA, Gd HP-DO3A, Mn DPDP, etc. or a nuclear agent such as ^{99m}Tc, ¹²⁵I, Ceretec, Myoview or Medronate II Technetium, all available from Nycomed Amersham), particulate agents may also be used if these are smaller than the particle size necessary to generate emboli (e.g., gas filled vesicles, iodinated organic compound containing vesicles, superparamagnetic particles or gadolinium oxalate particles, etc).

Furthermore, in the method of the invention a cytotoxic agent will preferably be administered before, with, or after the embolus generating particles. Chemoembolization is an established technique and a range of suitable cytotoxic agents is known, e.g., carboplatin, mitoxantrone, epirubicin, mitomycin C, decarbazine, vinblastine, cisplastin, interferon, dactinomycin, hydroxyurea, carmustine, methyl CNNU, interleukin-2, cyclophosphamide, amsacrine, doxorubicin, etc. This agent may be used at conventional cytotoxic doses (see for example Ryder et al. Gut 38: 125-128 (1996), Bedikian et al. Cancer 76: 1665-1670 (1995), Bronowicki et al. Cancer 74: 17-24 (1994) and Bartolozzi et al. Radiology 197: 812-818 (1995)). In one preferred embodiment of the invention, the particulate embolus generating agent also contains a cytotoxic agent, preferably a poorly water soluble compound, e.g., within the pores of a porous particle or as a surface coating on an insoluble contrast agent particle. Hydroxyapatite particles of greater than 75 micron diameter have been loaded with cytotoxins for prolonged release for therapeutic benefit (M. Imamura et al., Oncology Reports 2: 33-36 (1995) and K. Yamamura and T. Yotsuyanagi, Internat. J. Pharmaceutics 79: R1-R3 (1992)); however, their use as embolic agents was not disclosed in those reports. These drug delivery conjugates were implanted directly into tumors for treatment (K. Kunieda et al., Br. J. Cancer 67: 668-673 (1993)). The invention herein uses the hydroxyapatite as an embolic agent which can also have cytotoxins or other drugs adsorbed onto the particle surface. In this way the cytotoxic agent is released gradually from the particle following embolus formation so as to achieve an enhanced cytotoxic effect deriving from blood flow stoppage, from the released cytotoxic agent, and from the cytotoxic agent delivered before embolus formation occurred. As an alternative to a cytotoxic agent, a radio-pharmaceutical can be used with therapeutic intent. Lastly, it is worth noting that the successful delivery of peptide and protein derived therapeutic moieties is a difficult process, often involving enzymatic and hydrolytic degradation of the peptide/protein, with concomitant potential immunogenic effects and less than optimal biodistribution after injection. Adsorption onto or within the hydroxyapatite particles of the invention will minimize these negative effects because particle delivery is via catheter directly to the desired site of action, and thus exposure to plasma resident enzymes is minimized and there is limited exposure of the peptide/protein to the systemic immune system. Thus, embolic delivery of peptides and proteins offers many advantages over conventional injectable formulations.

In addition to trapping cytotoxins within tumors, the capillary embolic agents disclosed in this invention can also be used to temporarily or fractionally trap promoters of vascular growth, for example vascular endothelial growth factor (VEGF), vascular endothelial growth factor-related protein, basic fibroblast growth factors (bFGF and FGF-3), epidermal growth factor, hepatocyte growth factor, insulin-like growth factor, placental growth factor, placental proliferin-related protein, platelet-derived growth factor, platelet-derived endothelial growth factor, proliferin, proliferin-related protein, transforming growth factors α and β, tumor growth factor α. Naturally, agents disclosed in this regard can trap one or more of said promoters of vascular growth. See S. Strömblad and D. A. Cheresh, Cell Adhesion and Angiogenesis, Trends in Cell Biology 6:462-468, the disclosure of which is incorporated by reference. Preferred vascular growth promoters are VEGF, bFGF and FGF-3. In the instance where a tissue has suffered a decrease in blood supply due to an ischemic event, a vascular growth promoter can be delivered to promote the growth of new blood vessels (i.e., angiogenesis) which can reperfuse the tissue of interest. This can be accomplished with the current invention by fractionally embolizing the tissue (i.e., <5%) which would not significantly deny the tissue remaining blood flow but which would trap the vascular growth promoter in the location where it would generate badly needed new vessels. Alternatively, the capillary embolic agent could be generated from a temporary material (i.e., rapidly degraded) which would also trap the vascular growth promoter in the region of interest but would disappear due to kinetics rather than dependance upon fractional embolization.

Similarly, a vascular growth inhibitor could be trapped in an area, for example a neovascularizing tumor, where it is desirable to inhibit growth of new vasculature. Examples of such vascular growth inhibitors include tecogalan sodium (Daiichi), AGM-1470 (Takeda/Abbott), CM101 (Carbomed), mitaflaxone (Lipha), GM-1603 (Glycomed), rPF4 (Repligen), MPF-4 (Lilly), recombinant angiostatin (Entremed), endostatin, thalidomide (Entremed), DC101 (ImClone Systems), OLX-514 (Aronex), raloxifene hydrochloride (Lilly), suramin sodium (Parke-Davis), IL-12 (Roche), marimastat (British Biotech), and CAI (NCI). Naturally, agents disclosed in this regard can trap one or more of said inhibitors of vascular growth. A description of these compounds can be found in M. Barinaga, Designing Therapies that Target Tumor Blood Vessels, Science 275: 482-484 (1997) and “Antiangiogenic Agents”, The Year's New Drug News 1995: 601-603, the disclosures of which are herein incorporated by reference.

The embolic agents and pharmaceutical formulations thereof can be used as sensitizing agents for other therapeutic interventions, for example radiation, hyperthermia, or photolytic therapy. Materials useful as radiosensitizing agents are generally considered to be those materials which in an aqueous medium (i.e., intra or intercellular distribution) generate hydroxy radicals upon exposure to X-ray radiation. The energetic free radicals then react with cellular components and thereby effect a cytotoxic outcome. In addition, it could be expected that certain materials will become elevated in temperature as they absorb X-rays with or without generation of hydroxy radicals and thereby effect a cytotoxic outcome through the excess heat released from the agent within the local environment (i.e., thermolytic therapy). A more novel approach to radiation sensitization involves those agents which enhance radiation therapy by virtue of their ability to absorb X-rays and emit high energy particles, causing local cell damage and/or death. This effect can be achieved either via the insoluble particles, the soluble conventional contrast agent, or both. Preferred radiosensitizing agents are, for example, iodinated contrast agents such as NC 67722 (6-(ethoxycarbonyl)hexyl-bis(3,5-acetylamino)-2;4,6-triiodobenzoate), NC 12901 ((ethoxycarbonyl)methyl-bis(3,5-acetylamino)-2,4,6-triiodobenzoate), NC 70146 (1-(ethoxycarbonyl)pentyl-bis(3,5-acetylamino)-2,4,6-triiodobenzoate) or NC 8883 (ethyl-bis(3,5-acetylamino)-2,4,6-triiodobenzoate), or other radiodense material such as gadolinium oxide, gadolinium oxalate, and manganese-doped hydroxyapatite. The source of therapeutic radiation can be external to the tissue containing the embolic agent, for example from conventional radiation therapy equipment, or internal to the tissue containing the embolic agent, as in brachytherapy, for example from an implanted iodine source (for example RapidStrand or ¹²⁵I seeds manufactured by Nycomed Amersham), or from a device such as the Radiosurgery system of PhotoElectron Corp. of Lexington, Massachusetts, USA, which consists of a thin probe insertable into a tumor which emits therapeutic radiation from its tip. It is within the knowledge of one skilled in the art of radiation therapy to determine the appropriate therapeutic dose of radiation required for a particular patient with a particular condition.

In a further preferred embodiment, the particulate embolus generating agent also contains a biotherapeutic or targeted biotherapeutic moiety, for example the angiogenesis-inhibitor as proposed by Okada et al. in U.S. Pat. No. 5,202,352, antisense nucleic acids, diptheria toxin or ricin A chain.

The embolus generating agents used according to the invention will have a particle size appropriate for embolus generation in the target tissue of interest. For embolus formation in capillaries, the particle size may be in the range 1 to 50 μm, preferably 5 to 20 μm, i.e., much smaller than traditional embolus generating particles which generally serve to block a feeding artery for a tumor rather than the capillary vessels of the tumor itself. By blocking the capillaries using the particles according to the invention, the likelihood of collateral bypass of the intended embolization is reduced. Whilst contrast effective particles with sizes up to about 8 μm which can pass through the capillary are known as diagnostic imaging contrast agents, the small capillary blocking contrast effective particles are novel and they and pharmaceutical compositions thereof form a further aspect of the present invention. It should be noted that the larger known diagnostic agent particles with sizes above 5 μm are flexible particles which can deform to transit the capillaries—the capillary blocking particles of the present invention will on the other hand be inflexible particles when the particle size is towards the bottom of the 1 to 50 μm range, preferably the 5 to 20 μm range, e.g., at 20 μm or below, preferably at 12 μm or below.

In general, for capillary embolization, the particle size will preferably be 5 to 25 μm, especially 10 to 20 μm, more especially 7 to 15 μm, and most especially 8 to 12 μm.

Alternatively, smaller particles, of a size normally associated with use as diagnostic imaging contrast agents can be used as embolus forming agents if components normally added to their pharmaceutical formulations so as to prevent aggregation or gel formation are omitted or used in reduced concentrations. Thus one may use particles conventionally thought to be too small to cause emboli, as they are capable of passing through the capillary beds. However, the formulation of these agents is such that they can be prepared and sterilized and be physically and chemically stable, yet upon exposure to the biological environment of blood or tissues particles either aggregate or gel to form the desired emboli. While the emboli creation can be controlled by particle size, the formulation is also important in the formation of the emboli. For example, conventional nanoparticle surfactants such as the Pluronics may be omitted from these formulations since they retard aggregation and gel formation and hence afford a greater probability that emboli may form elsewhere in the body other than the desired site.

Thus in such formulations, particles which would be thought to be capable of passing through the capillary beds by virtue of their size will still qualify as embolus-forming contrast effective particles.

The method of the invention may also be used to block larger blood vessels, e.g., the larger feeding arteries leading to the tissue site of interest (e.g., a tumor or a site intended for surgical intervention) and in this case larger embolus generating particles may be used, e.g., having particle sizes up to 2 mm, preferably from 50 to 1500 μm, especially about 100 μm. Pharmaceutical compositions containing such large (>20 μm, preferably >40 μm) particles in a physiologically tolerable sterile aqueous carrier medium are also novel and form a further aspect of the invention.

Appropriate particle sizes can be achieved by size separation of polydisperse particle mixtures, by milling, or by the use of core particles of appropriate size, controlled by precipitation or crystallisation: Milling can include dry milling, jet milling, wet milling or any other particle size enhancement via attrition processes. In addition a microfluidizer can be used to disperse and prepare these particle suspensions via the shear and impact of that process. This is controllable by the number of passes (i.e., residence time) and the applied pressure. There are a number of particle preparation procedures which can be used to control the size of the core including thermolysis of solutions or suspensions, evaporative precipitation and ultrasonic dispersion.

The embolus generating particles used according to the invention are preferably capable of being broken down in vivo, either over a prolonged period of time or relatively rapidly once the need for the embolus is removed (e.g., following the surgical intervention when an embolus has been created to reduce bleeding during an operation). However permanent embolization can also be achieved with embolic agents according to the invention, e.g., agents which are poorly biodegradable.

For relatively large embolus generating particles particle breakdown can be achieved by laser lithotripsy, by guiding a light transmitting fibre to the particle and subjecting the particle or the immediately adjacent plasma to a burst of light energy. For smaller particles, alternative breakdown mechanisms are necessary. For gas containing vesicles, a high energy pulse of ultrasound may be used to burst the particles and remove the blockage to blood flow. Indeed this technique may be used downstream of the embolization site during embolus formation to destroy any embolus generating particles that are not retained at the target site and so prevent unwanted emboli from being formed elsewhere. The technique similarly may be used to enhance cytotoxic drug delivery where smaller gas and cytotoxic agent containing particles are administered to create capillary emboli followed by larger gas-free particles to cause further upstream embolization. Subsequent to location of both sets of particles, the smaller particles may be burst by ultrasound to release the cytotoxic agent in a flow free zone in or adjacent the tumor.

Alternative methods of ensuring breakdown of the embolus generating agent include the use of contrast agents or coatings (or vesicle membranes) which while effectively water-insoluble, are biodegraded, e.g., due to the presence of ester bonds or other biodegradable linkages. It is also feasible that other interventional techniques can be used to remove the emboli, such as surgical resection and other removal techniques viz vacuum removal.

The embolus generating particles may be formulated for administration together with conventional pharmaceutical excipients or other ‘active’ agents, including for example: soluble or capillary transiting contrast agents (as discussed above); cytotoxic agents (as discussed above); liquid carrier media (e.g., pyrogen free water, saline, water for injections and ethanol); salts (e.g., of plasma cations with physiologically tolerable counterions), sugars, sugar alcohols and other osmolality adjusting agents; viscosity modifiers, emulsifiers and stabilizers; buffers and pH adjusting agents; polyethylene glycols, etc.

It is thought that iso-osmotic preparations or slightly hyperosmotic preparations will function better than hypo-osmotic suspensions, although all appear to work well.

The particle concentration and the dosage will depend upon the patient, the selected particle size, the intended embolization location and the administration route. Since administration will generally be via injection, preferably via a catheter, upstream of the intended embolization location, the number of particles required will clearly be dependent upon the number of paths downstream of the injection site which are capable of being blocked by the particles. However particle concentrations will preferably be below 20% wt/vol in the overall compositions and more preferably below 10% and where a soluble contrast agent is included in the carrier medium this will preferably be at a concentration of less than 10% wt/vol for MRI but greater than 5% for CT and more preferably greater than 20% as soluble agents will be viewed by fluoroscopy which is less sensitive than CT and requires increased agent.

Thus by way of example, in animal experiments, 50 μL of a 10% suspension of capillary embolic agent was effective for rat brain whereas 100-250 μL of the same suspension was effective for myocardium or kidney.

The particles of the invention may particularly suitably be used to reduce actual or anticipated blood-leakage (e.g., during surgery), and in embolization and chemoembolization therapy of tumors, particularly heptatocellular carcinomas, head and neck tumors, uterine tumors, renal tumors and other solid tumors.

The embolic agent used according to the invention preferably comprises particles already of a size appropriate to cause embolus formulation at the desired site. As an alternative however, the invention may involve administration of a composition which is reactive with body fluid (e.g., blood) to produce particles of the appropriate size and composition. This represents a further aspect of the invention. In this aspect, the composition will contain a biologically compatible liquid solvent system (e.g., containing an alcohol, ester, ether DMSO or dimethyl formamide, and limited aqueous mixtures of such solvents, for example a 1:1 mixture of DMSO and water) and a particle forming or particle enlarging agent which is less soluble in the body fluid than in the liquid solvent system. Such an agent may be a biologically compatible polymer which enlarges particles of an organic iodinated diagnostic agent or agents present in the composition or which forms particles entrapping such iodinated organic agent or agents in solution in the liquid solvent system. Alternatively, the particle forming agent may be a diagnostically effective agent which is soluble in the liquid solvent system but forms particles or droplets on contacting body fluids such as blood. A biologically active molecule can be included in the formulation such that the biologically active molecule is trapped within the tissue vasculature after embolization and stoppage of blood flow. The biologically active molecule can be, for example, a cytotoxin, a biotherapeutic, or a targeted biotherapeutic all as described above, an anti-inflammatory agent, etc.

Examples of such diagnostically effective agents include the water insoluble or poorly water soluble solid or liquid iodinated organic compounds disclosed in the patent publications of the 1990's from Sterling Winthrop Inc.

Iodinated agents which could be useful in such applications include degradable agents containing labile ester functionalities and agents which are essentially not degradable. Additionally, agents which are oils and are soluble in the solvent system for the polymer can be used. While these oils are not particulates, they are water insoluble and inert with respect to degradation and would be captured within the precipitating polymer as the embolus is formed.

In this aspect of the invention, the composition before administration preferably is free of particles, i.e., the diagnostic agent is preferably soluble in the liquid solvent system.

The benefit of this formulation is that it does not require particles per se in the dosage form thereby obviating any problems within the catheter during dosing due to aggregation, etc. This improvement also extends to the shelf stability of the embolic agent with respect to settling of the contrast gents and aggregation, resuspension, etc. Also, the organic nature of these agents may make them much more compatible with the precipitating polymers serving to bind the various polymer segments together for a more permanent blockage. Lastly, some of these agents have demonstrated excellent safety upon iv injection (as nanoparticle suspensions or oil-in-water emulsions) suggesting that they would have an advantage over materials like barium sulfate and other inorganic particles.

Patents and other publications referred to herein are hereby incorporated by reference.

The invention will now be illustrated further with reference to the following non-limiting Examples in which parts, percentages and ratios are by weight unless otherwise specified.

### EXAMPLE 1

NC 12901: Ethyl (3,5-diacetamido-2,4,6-triiodobenzoyloxy)acetate (U.S. Pat. No. 3,097,228)

A mixture of 63.6 g (0.1 mol) of sodium diatrizoate and 14.7 g (0.12 mole) of ethyl chloroacetate in 175 ml of dimethylformamide was heated on a steam bath with stirring for six hours. The reaction mixture was filtered while-hot and the filtrate was diluted with cold water to a volume of 500 ml. The solid material which had separated was collected by filtration and stirred with 500 ml of 5% sodium bicarbonate solution. The product was again collected by filtration, washed with water, followed by ether and then dissolved in 300 ml of hot dimethyl formamide. The resulting solution was filtered, diluted with 350 ml of hot water and cooled. The resulting product was collected by filtration and dried to give 53 g of ethyl (3,5-diacetamido-2,3,6-triiodobenzoyloxy)acetate, mp 269.5-270.5° C. (dec.).

Calculated for C₁₅H₁₅I₃N₂O₆: C, 25.73; H, 2.15; 154.4; Found: C, 25.80; H, 2.77; I 53.9.

### EXAMPLE 2

Embolization Composition

A 20 ml slurry of NC 12901 was prepared using 2.0 g of NC 12901 and 1.0 g of iohexol (solid) in 18.31 ml of water. This slurry was added to a 1 oz brown glass bottle along with 15 ml of 1.1 mm diameter zirconium silicate milling beads. The resulting slurry was 10% NC 12901 and 5% iohexol (wt/vol %). This slurry was rolled at approx. 100 rpm overnight. At the end of that time, the slurry had been transformed into a white, milky suspension. The suspension was separated from the milling beads by pipetting or by filtration through coarse mesh screen. Particle size was determined by light scattering using a Horiba 910a particle sizing instrument. After milling, the average particle size was determined to be 3.96 microns with a broad standard deviation of 2.56 microns. After autoclaving, the average particle size was determined to be 8.10 microns, again with a broad particle size distribution of 3.90 microns. These large particles settle slightly with time but are easily resuspended with gentle shaking.

### EXAMPLE 3

Embolization Composition

A 20 mL suspension in water of 10% NC 12901 and 10% iohexol (wt/vol %) was added to a 1 oz amber wide mouth bottle containing 15 mL preconditioned 1.1 mm ZrSiO₃ beads such that the bottle was just full to the top. Care was taken to minimize or remove any head space from the bottle. The entire 20 ml suspension did not fit into the jar with the milling beads and some of the suspension was not milled and thus was discarded. The sample bottle was allowed to roll on a US Stoneware 3 tiered roller mill (East Palestine, Ohio) at approximately 125 rpm for 24 hours. At the end of this time, the suspension was separated from the milling beads by pipetting or by filtration through a coarse mesh screen. Particle size and pH were measured using the Horiba LA910 (Irvine, Calif.) particle-size analyzer and a standard digital pH meter. The average particle size was 2.6 μm. Samples were diluted in 0.001% dioctyl sulfosuccinate for size measurement. The harvested suspensions were then autoclaved for 15 minutes at 121.1° C. in standard crimp sealed glass vials at half fill. The particle size and pH were measured after autoclaving. The average particle size was 5.6 μm.

### EXAMPLE 4

NC 8883—Ethyl 3,5-diacetamido-2,4,6-triiodobenzoate

To 8.11 L of dry dimethylformamide was added 1.01 kg (1.65 mole) of diatrizoic acid. To the vigorously stirred suspension was carefully added 274 g (1.99 mol) of milled potassium carbonate. During the addition, there was significant gas evolution. Before all of the solid had gone into solution, a second solid began to form at the end of the carbonate addition. The mixture was stirred for 30 minutes at room temperature followed by the dropwise addition of 608 g (3.90 mole) of ethyl iodide. After stirring overnight, the mixture had become essentially homogeneous and was poured into 25 L of water. The resulting precipitate was collected by filtration, washed thoroughly with water, and dried under vacuum at 60° C. to afford 962 g (91%) of ethyl 3,5-diacetamido-2,4,6-triiodobenzoate as a white solid, mp 280-290° C. (dec.).

Calculated for C₁₃H₁₃I₃N₂O₄: C, 24.32; H, 2.05; N, 4.36; Found: C, 24.27; H, 1.93; N, 4.28.

### EXAMPLE 5

Embolization Composition

Example 3 was repeated using NC 8883 in place of NC 12901. The average particle size before autoclaving was 5.4 μm while after autoclaving the average size was 15.0 μm.

### EXAMPLE 6

Embolization Composition

A 1 oz amber wide mouth bottle was rinsed with NanoPure water several times. The cap was rinsed with 70% isopropyl alcohol followed by NanoPure water and set aside. The bottle was filled with 15 mL preconditioned 1.1 mm zirconium silicate beads, covered with aluminum foil and depyrogenated for 8 hours at 240° C. All other glassware necessary to prepare surfactant, excipient or buffer solutions was depyrogenated. Any other remaining equipment was autoclaved. A 20 mL suspension in NanoPure water of 10% NC 12901 and 10% iohexol was prepared using solutions prepared by aseptic technique and filtered through sterile filters (i.e., 0.2 micron Acrodisc® filter). The bottle was filled to capacity such that no air head space was present. The bottle was sealed with the above cleaned cap and roller milled for 24 hours. After milling was completed, the suspension was harvested into sterile (i.e., rinsed and autoclaved) glass vials without further dilution and sealed with standard Teflon lined stoppers. The sealed vials were the autoclaved for 15 minutes at 121.1° C. Particle size, pH and osmolality were measured and recorded on extra samples prepared in parallel for testing.

### EXAMPLE 7

Embolization Composition

Example 6 was repeated using NC 8883 in place of NC 12901.

### EXAMPLES 8 AND 9

Examples 6 and 7 respectively were repeated using 5% iohexol in place of 10% iohexol.

The particle sizes for the compositions of Examples 6 to 9 were as follows:

| | | Average particle size |
|---|---|---|
| | Dosage | (μm) |
| | before | After |
| Example | Autoclaving | Autoclaving |
| 6 | 2.6 | 5.6 |
| 7 | 5.4 | 15.0 |
| 8 | 5.9 | 8.1 |
| 9 | 4.3 | 6.6 |

### EXAMPLE 10

Embolus Formation in the Rabbit

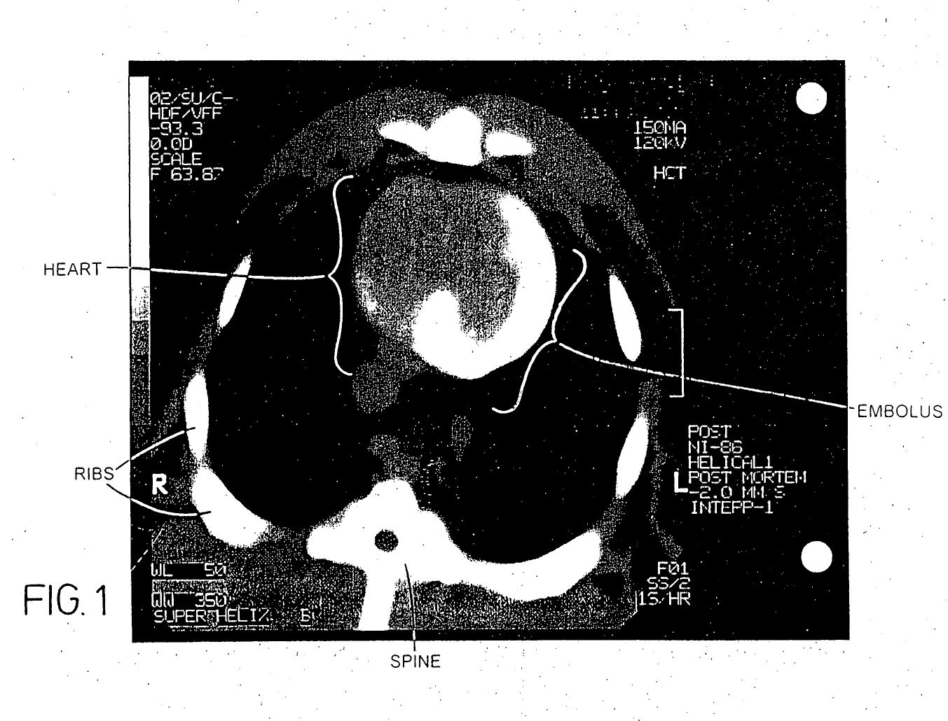

0.1 mL of the composition of Example 6 was injected into the circumflex coronary artery of the rabbit to create emboli in the myocardium. After 10 minutes an X-ray CT image of the rabbit was recorded. This image, FIG. 1 of the accompanying drawings, shows the selective embolization of the myocardium that was achieved.

### EXAMPLE 11

The Formulation of Nanoparticles as Embolic Agents

A suspension of NC 70146 is prepared by adding 22.5 gm (22.5%, wt/vol %) of NC 70146 to a brown glass vial together with 4.5 gm (4.5%, wt/vol %) of biolpaque (NC 8851) and approximately 87 ml of water. Enough 1.1 mm zirconium silicate milling beads is added to fill the glass jar halfway and the suspension is milled for three days at 150 rpm. At the end of this time, the particles are pipetted away from milling beads and sized at approximately 100 nm in average diameter using the Horiba 910a particle sizing instrument. After autoclaving, these beads are approximately 150 nm in average particle size.

Upon addition of this suspension to plasma or whole blood, the suspension will aggregate and gel, forming a clot within the plasma or blood stream and thus giving rise to an embolus-forming suspension.

### EXAMPLE 12

6 to 10 Micron Particles of Hydroxyapatite Prepared with Tohexol for Embolization

A 20 ml slurry of hydroxyapatite was prepared using 1.0 g of hydroxyapatite and 7.6 g of iohexol (solid) in 15.31 ml of water. This slurry was added to a 1 oz brown glass bottle along with 15 ml of 1.1 mm diameter zirconium silicate milling beads. The resulting slurry is 5% hydroxyapatite and 38% iohexol (wt/vol %). This slurry was rolled at approximately 100 rpm overnight. At the end of that time, the slurry had been transformed into a white, milky suspension with a pH=7.28. Particle size was determined by light scattering using a Horiba 910a particle sizing instrument. After milling, the average particle size was determined to be 7.3 microns with a broad standard deviation of 5.4 microns. After autoclaving, the average particle size was determined to be 7.1 microns, again with a broad particle size distribution of 4.2 microns. As observed before with NC 12901, these large particles settle with time but are easily resuspended with gentle shaking.

These particles were examined in an acute pig model where 3.0 ml of suspension was administered directly into the renal artery by surgical cutdown on an anesthesized pig. Blood flow was then reestablished for 10 minutes before the kidney was imaged by conventional X-ray at 50 kV and-2 mamps. The X-ray clearly showed the complete kidney to be embolized by the agent. In addition, by comparison with a hydroxyapatite suspension without the iohexol added in the other kidney, it was further clear that the embolus traps the iohexol within the tissue. Thus, the drug delivery aspects of this embolic agent to the embolized tissue are confirmed (ie as iohexol can be delivered, so too can other “drugs”)

### EXAMPLE 13

Demonstration of the Embolic Effects of very small Particles—NI 70

A particle suspension was prepared as in Example 11 using NC 67722, 6-(ethoxycarbonyl)hexyl-bis(3,5-acetylamino)-2,4,6-triiodobenzoate (synthesis described in U.S. Pat. No. 5,322,679). After milling, the suspension was filtered through a 0.2 micron sterile filter and determined to have an average particle size of 91 nm (std. dev. 23 nm) using light scattering (Horiba 910a). The osmolality of this suspension was 304 mOsm/kg while the pH=8.0. Upon administration to a rabbit intravenously via the ear vein, a massive embolization of the pulmonary vessels was achieved within 30 seconds. The embolization was confirmed by CT X-ray imaging of the rabbit. Thus, very small particles, when formulated as embolic agents, can act efficiently to embolize the vascular system from the injection site.

### EXAMPLE 14

Embolic Particles of NC 8883 prepared with 43% Iohexol for Embolization (NI 239 and NI 243)

A 15 ml slurry of NC 8883 was prepared using 1.5 g of NC 8883 and 1.98 ml of Omnipaque 350 (i.e., 76% iohexol) in 12.4 ml of Nanopure™ water. This slurry was added to a 60 ml brown glass bottle along with 30 ml of 1.1 mm diameter zirconium silicate milling beads. The resulting slurry is 10% NC 8883 and 10% iohexol (wt/vol %). This slurry was rolled at approximately 100 rpm overnight. After 24 hours, the slurry was recovered and diluted by a factor of 2 with 76% iohexol (i.e., Omnipaque 350) such that the final formulation was 5% NC 8883 and 43% iohexol. The final suspension was then autoclaved at 121° C. for 15 minutes. After autoclaving, the pH=7.3 and the average particle size as determined by light scattering (Horiba 910a) was determined to be 7.2 microns with a standard deviation of 3.7 microns. As observed before with earlier embolic formulations, these large particles settle with time but are easily resuspended with gentle shaking.

These particles were examined in a rabbit kidney model for efficacy by digital subtraction angiography (DSA) and conventional CT X-ray imaging. A small catheter was guided into the renal artery of the rabbit whereupon 0.30 ml of embolic agent was injected through the catheter. The arterial bed of the kidney was seen to immediately opacify by DSA and to remain opacified during the course of the imaging session. 4th level arteries could be observed during the imaging process. CT imaging confirmed the presence of X-ray dense material within the kidney showing uniform opacification of the entire kidney cortex region. These data confirm the complete embolization of the kidney in the rabbit and the X-ray opacification via DSA and the more sensitive CT imaging modalities.

### EXAMPLE 15

Embolic Particles of NC 8883 Prepared with 10% Iohexol for Embolization of the Rabbit Kidney

A slurry of NC 8883 was prepared as in Example 7 with an average particle size of 15 microns. The resulting slurry was then examined in the rabbit kidney as in Example 14 affording excellent CT X-ray enhancement of the renal arterial bed confirming the embolization of this tissue at lower values of added iohexol (i.e., 10%)

### EXAMPLE 16

Embolic Particles of Hydroxyapatite Prepared with 2.55% Mannitol for Embolization (NI 250)

A 30 ml slurry of hydroxyapatite (HA) was prepared using 3 g of HA and 1.53 g of mannitol in 26 ml of water. The HA used in this preparation was purchased from AIC (American International Chemical, Natick, MA, lot# ABB2804) with an average particle size of 20 microns. The suspension was homogenized using an Ultra Turrax T-25 tissue disrupter (IKA Laboratories) for 10 minutes at a speed of 24000 rpm. 5 ml of water for injection was then added to the suspension making the final concentrations 5% HA and 2.55% mannitol. The suspension was then sterilized by conventional steam sterilization at 121° C. for 20 minutes. After autoclaving, the average particle size was determined to be 7.5 microns (std. dev. 3.8 micron) with a range of 2 to 30 microns using light scattering (Horiba 910a). The suspension was also determined to have a pH=7.5 and osmolality=179 mOsm/kg.

### EXAMPLE 17

Embolic Particles of Hydroxyapatite Prepared with 2.55% Mannitol and 38′ Iohexol for Embolization (NI 251)

A 30 ml slurry of hydroxyapatite (HA) was prepared using 3 g of HA and 1.53 g of mannitol in 26 ml of water. The HA used in this preparation was purchased from AIC (American International Chemical, Natick, Mass., lot # ABB2804) with an average particle size of 20 microns. The suspension was homogenized using an Ultra Turrax T-25 tissue disruptor (IKA Laboratories) for 10 minutes at a speed of 24000 rpm. 5 ml of Omnipaque 350 (i.e., 76% iohexol) was added to the suspension making the final concentrations: 5% HA, 2.55% mannitol, and 38% iohexol. The suspension was then sterilized by conventional steam sterilization at 121° C. for 20 minutes. After autoclaving, the average particle size was determined to be 7.0 microns (std. dev. 3.7 micron) with a range of 2 to 30 microns using light scattering (Horiba 910a). The suspension was also determined to have a pH=7.4 and osmolality=599 mOsm/kg.

### EXAMPLE 18

Embolic Particles of NC 67722 Prepared with 2.5% Mannitol for Embolization (NI 252)

A 40 ml slurry of NC 67722 was prepared using 4 g of NC 67722 and 2.0 g of mannitol in 36.2 ml of water. The suspension was roller milled for 24 hours at approximately 150 rpm in a 60 ml bottle using 30 ml of 1.1 mm zirconium silicate milling beads. The suspension was harvested and sterilized by conventional steam sterilization at 121° C. for 15 minutes. An equal volume of water for injection was then added to the suspension making the final concentrations 5% NC 67722 and 2.5% mannitol. The average particle size was determined to be 16.8 microns (std. dev. 8.8 micron) with a range of 2.6 to 77 microns using light scattering (Horiba 910a). The suspension was also determined to have a pH=6.1 and osmolality=166 mOsm/kg.

### EXAMPLE 19

Embolic Particles of NC 67722 Prepared with 2.5% Mannitol and 38% Iohexol for Embolization (NI 253)

A 40 ml slurry of NC 67722 was prepared using 4 g of NC 67722 and 2.0 g of mannitol in 36.2 ml of water. The suspension was roller milled for 24 hours at approximately 150 rpm in a 60 ml bottle using 30 ml of 1.1 mm zirconium silicate milling beads. The suspension was harvested and sterilized by conventional steam sterilization at 121° C. for 15 minutes. An equal volume of Omnipaque 350 (i.e., 76% iohexol) was then added to the suspension making the final concentrations 5% NC 67722, 2.5% mannitol, and 38% iohexol. The average particle size was determined to be 12.0 microns (std. dev. 6.2 micron) with a range of 1.7 to 51 microns using light scattering (Horiba 910a). The suspension was also determined to have a pH=7.3 and osmolality=606 mOsm/kg.

### EXAMPLE 20

Embolic Particles of NC 8883 Prepared with 2.5% Mannitol for Embolization (NI 254)

A 40 ml slurry of NC 8883 was prepared using 4 g of NC 8883 and 2.0 g of mannitol in 36.3 ml of water. The suspension was roller milled for 24 hours at approximately 100 rpm in a 60 ml bottle using 30 ml of 1.1 mm zirconium silicate milling beads. The suspension was harvested and sterilized by conventional steam sterilization at 121° C. for 15 minutes. An equal volume of water for injection was then added to the suspension making the final concentrations 5% NC 8883 and 2.5 mannitol. The average particle size was determined to be 21.8 microns (std. dev. 20 micron) with a range of 2.3 to 77 microns using light scattering (Horiba 910a). The suspension was also determined to have a pH=6.4 and osmolality=172 mOsm/kg.

### EXAMPLE 21

Embolic Particles of NC 8883 Prepared with 2.5% Mannitol and 38% Iohexol for Embolization (NI 255)

A 40 ml slurry of NC 8883 was prepared using 4 g of NC 8883 and 2.0 g of mannitol in 36.3 ml of water. The suspension was roller milled for 24 hours at approximately 100 rpm in a 60 ml bottle using 30 ml of 1.1 mm zirconium silicate milling beads. The suspension was harvested and sterilized by conventional steam sterilization at 121° C. for 15 minutes. An equal volume of Omnipaque 350 (i.e., 76% iohexol) was then added to the suspension making the final concentrations 5% NC 8883, 2.5% minnitol and 38% iohexol. The average particle size was determined to be 14.4 microns (std. dev. 6.5 micron) with a range of 1.3 to 45 microns using light scattering (Horiba 910a). The suspension was also determined to have a pH=7.4 and osmolality=579 mosm/kg.

### EXAMPLE 22

Embolic Particles of Hydroxyapatite Prepared with 38% Iohexol for Embolization Without Milling or Homogenization (NI 265)

A 50 ml slurry of hydroxyapatite (HA) was prepared using Sg of HA and 48.4 ml of water. The HA used in this preparation was purchased from AIC (American International Chemical, Natick, Mass., lot # 11A1810A (H10PJC)) with an average particle size of 10 microns. An equal volume of Omnipaque 350 (i.e., 76% iohexol) was then added to the suspension making the final concentrations 5% HA and 38% iohexol. The suspension was then sterilized by conventional steam sterilization at 121° C. for 15 minutes. After autoclaving, the average particle size was determined to be 12.5 microns (std. dev. 5.4 micron) with a range of 3 to 39 microns using light scattering (Horiba 910a). The suspension was also determined to have a pH=5.86 and osmolality=348 mosm/kg.

### EXAMPLE 23

Embolic Particles of Hydroxyapatite Prepared with 38% Iohexol for Embolization Without Milling or Homogenization and with 2.8 mg/ml of Omniscan (NI 264)

0.1 ml of Omniscan (287 mg/ml) was added under sterile conditions to 10 ml of a suspension of hydroxyapatite as prepared in Example 22. The average particle size was determined to be 12.6 microns (std. dev. 5.5 microns) with a range of 3 to 39 microns using light scattering (Horiba 910a). The pH was determined to be 5.9 and the osmolality was found to be 366 mOsm/kg.

### EXAMPLE 24

Embolic Particles of Hydroxyapatite Prepared with 38% Iohexol for Embolization Without Milling or Homogenization and with 28.7 mg/ml of Omniscan (NI 269)

10 ml of hydroxyapatite (HA) suspension as prepared in Example 22 was allowed to settle such that 1 ml of the clear supernatant was removed and replaced with 1 ml of Omniscan (287 mg/ml). After autoclaving the average particle size was determined to be 12.1 microns (std. dev. 5.4 microns) with a range of 3 to 39 microns using light scattering (Horiba 910a). The pH was determined to be 5.9 and the osmolality was found to be 408 mOsm/kg.

### EXAMPLE 25

Embolization of a Perfused Rat Liver with Hydroxyapatite Embolic Particles

An embolic particle suspension of hydroxyapatite (HA) as prepared in Example 22 was tested in an isolated perfused rat liver model. The rat was anaesthetized with 50 mg/kg of sodium pentobarbital i.p. A surgical incision was made at the midline of the ventral side of the abdomen to expose the liver. The liver was cannulated via the portal vein and then perfused with Krebs-Henseleit buffer which was saturated with gas at 95% oxygen and 5% carbon dioxide at a flow rate of 2 to 4 ml/min/g tissue weight in a single pass setup. The inferior vena cava and bile duct were cannulated with a catheter for monitoring the venous outflow and bile flow, respectively. The animal was humanely sacrificed at 37±1° C. During the perfusion, the oxygen consumption and intrahepatic pressure were monitored by a PO-NE-MAH system (Goup Instrument, Ohio)

The intrahepatic pressure immediately increased from 30 to 120 mm Hg after a bolus injection of 3.0 ml of the HA embolic suspension into the inflow of perfusate (dilution factor of 29×). Simultaneously, the oxygen consumption in the perfused liver was reduced to almost zero. The hepatic outflow from the inferior vena cava was completely stopped by administration of the HA. These results clearly indicate that a bolus injection of HA particles embolized the liver completely.

The liver was taken at the end of the study and fixed in 10% neutral buffered formalin. Randomly selected sections for microscopy were embedded in paraffin, sectioned at 5 microns, and stained with hematoxylin and eosin. Microscopic examination revealed variable filling of portal veins throughout the sections with irregularly sized and shaped basophilic particles of test article (HA). While occasional particles were found lodged in the liver sinusoids adjacent to a portal vein, in general, the particles did not pass into vascular spaces smaller than the portal veins. No particles were seen in the central veins exiting the liver lobules.

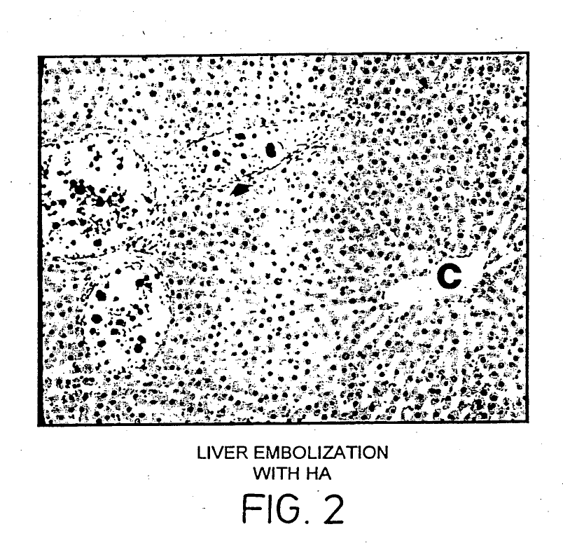

FIG. 2 of the accompanying drawings shows a photographed section of a rat liver which received HA by bolus injection into the main portal vein of an isolated perfused whole organ preparation. Multiple portal veins, which carry blood entering the liver lobules, are filled with hydroxyapatite crystals of varying sizes (arrow). An adjacent central vein (C), which carries blood out of the liver back to the general circulation, does not contain HA. FIG. 2 was obtained using Hematoxylin and Eosin (H&E) stain at 250× magnification.

### EXAMPLE 26

Embolization of a Perfused Rat Kidney with Hydroxyapatite Embolic Particle

An embolic particle suspension of hydroxyapatite (HA) as prepared in Example 22 was tested in an isolated perfused rat kidney model. The average particle size of this embolic suspension was determined to be 6.1 micron (std. dev.=2.0 microns) and a range of particle sizes from 2.3 to 17 microns. The rat was anaesthetized with 50 mg/kg of sodium pentobarbital i.p. A surgical incision was made at the midline of the central side of the abdomen to expose the kidney. The kidney was cannulated via the abdominal artery and then perfused with Krebs-Henseleit buffer which was saturated with gas at 95% oxygen and 5% carbon dioxide at a flow rate of 2 to 4 ml/min/g tissue weight in a single pass setup. Renal vein and ureter were cannulated through a catheter for monitoring the venous outflow and urine flow, respectively. The animal was humanely sacrificed under deep anaesthesia and the kidney was removed to an organ perfusion unit which was maintained at 37±1° C. During the perfusion, the oxygen consumption and intrarenal pressure were monitored by a PO-NE-MAH system (Goup Instrument, Ohio).

The intrarenal pressure immediately increased from 40 to 130 mm Hg after a bolus injection of 1.0 ml of the HA embolic suspension into the inflow of perfusate (dilution factor of 7×). Urine flow was completely stopped by administration of the HA as was the outflow of perfusate from the renal vein. These results clearly indicate that a bolus injection of HA particles embolized the kidney completely.

The kidney was taken at the end of the study and fixed in 10% neutral buffered formalin. Randomly selected sections for microscopy were embedded in paraffin, sectioned at 5 microns, and stained with hematoxylin and eosin. Microscopic evaluation revealed fine granular basophilic deposits of test article within the arcuate arteries and in vessels smaller than the arcuate arteries including the glomerular capillaries.

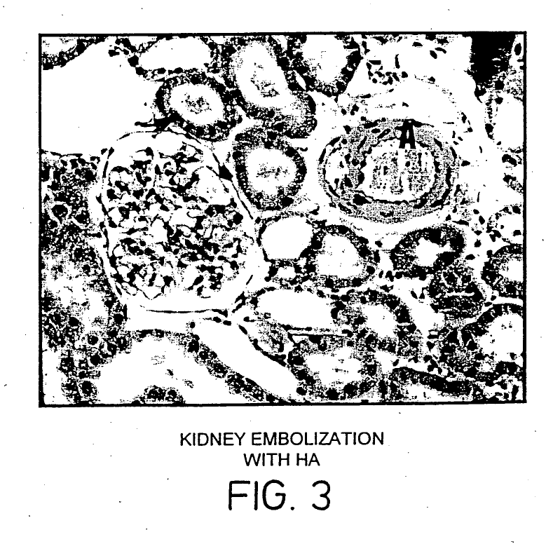

FIG. 3 of the accompanying drawings shows a photographed section of a rat kidney which received a finely ground formulation of HA by bolus injection into the renal artery of an isolated perfused organ preparation. Fine crystalline particles of HA are present within small arteries (A) in the renal cortex as well as in capillary loops in the glomeruli (arrows). FIG. 3 was obtained using Hematoxylin and Eosin (H&E) stain at 400× magnification.

### EXAMPLE 27

Embolization of a Rat Kidney with Hydroxyapatite Embolic Particles

The renal arteries of freshly killed rats were injected directly with hydroxyapatite (HA) embolic particles as prepared in Example 22. The volume injected varied, but averaged near 0.5 ml. The kidneys were then fixed in 10% neutral buffered formalin. Randomly selected sections for microscopy were embedded in paraffin, sectioned at 5 microns, and stained with hematoxylin and eosin. Microscopic evaluation showed excellent filling of the renal artery and arcuate arteries and variable filling of the smaller arterioles down to the size of the afferent arterioles of the glomeruli. In the latter case, a few particles were seen to be stopped at the capillary level of the glomeruli. No particles were seen to reach the venous system of the kidney.

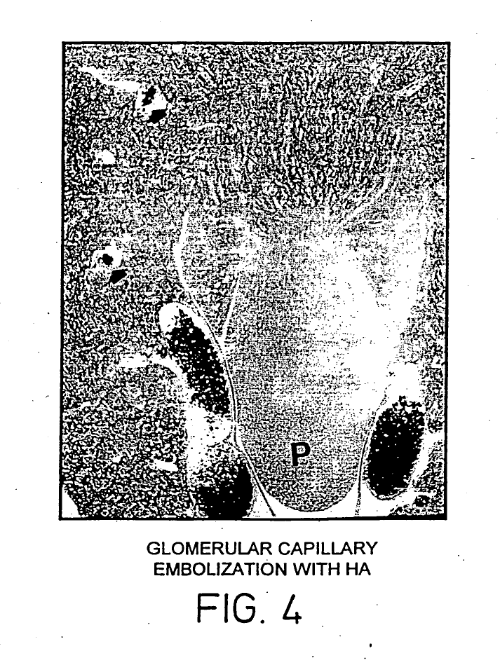

FIG. 4 of the accompanying drawings shows a photographed section of a rat kidney which received HA by direct injection into the renal artery. There is complete filling of the large renal arteries adjacent to the papilla (P), as well as filling of the smaller arcuate arteries (arrows), which supply blood to the cortex and medulla. FIG. 4 was obtained using Hematoxylin and Eosin (H&E) stain at 125× magnification.

### EXAMPLE 28

Embolization of Systemic Organs to Assess Regional Blood Flow

A rabbit was catheterized and the catheter positioned in the left ventricle to compare the proportional embolization of systemic organs with a capillary material such as is well known to reflect regional blood flow when radioactively labelled microsphere particles are arterially administered (A. M. Rudolph and M. A. Heyman, Circ. Res. 1967, vol 21, 163-184). A capillary embolic suspension as prepared in Example 6 was injected (2.0 ml) and allowed to circulate. Following sacrifice of the animal, the entire corpus was scanned with computed tomography (CT) X-ray. Tissues with abundant arterial perfusion, such as kidney cortex and myocardium had significant radiopaque enhancement as measured by the increase in Hounsfield Units of these tissues, while tissues with little capillary perfusion such as the renal medulla and the muscle had little enhancement. Liver perfusion, which comes predominantly from post capillary blood, was not noted in this experiment.

### EXAMPLE 29

Selective Embolization of the First Capillary Bed

To further demonstrate selective embolization of the first capillary bed, several anaesthetized rabbits were subject to selective catheterization of the main renal artery or to subselective catheterization of the dorsal or ventral branches. Embolization was accomplished with from 100 to 300 microliters of agents as prepared in Examples 14, 15 and 23. The injections were monitored with fluoroscopy or digital subtraction angiography. It was found that supplemental water-soluble contrast agent was required for visualization by the latter, but all these injections showed CT localization of the radiopaque emboli limited to the kidney cortex within the circulation of the injected artery. Thus, when the injection was into the main renal artery, all portions of the kidney were embolized in proportion to dose. When the injection was subselective, only that portion of the kidney was embolized. Further, the radiopaque emboli were limited to the renal cortex. No emboli were identified within the medulla or papillar, regions of the kidney that are supplied by post-capillary blood flow.

### EXAMPLE 30

Selective Embolization of the First Capillary Bed in the Kidney of the Pig

The renal artery embolization experiment was repeated in an anaesthetized pig. The left kidney was catheterized and injected with 2 ml of an agent as prepared in Example 20. The injection could not be seen by fluoroscopy or digital subtraction angiography, but postmortem CT revealed effective capillary embolization in the renal cortex, sparing the renal medulla. The right kidney was catheterized and injected with 2 ml of an agent as prepared in Example 14. Here the injection was easily observed by fluoroscopy and digital subtraction angiography. Again, CT scans showed embolization limited to the kidney cortex.

### EXAMPLE 31

VX2 Carcinoma in the Rabbit Kidney

A rabbit was prepared with VX2 carcinoma growing in the kidney. The experimental kidney was embolized with 2 injections of 300 microliters of an agent as prepared in Example 21. The rabbit was followed for 48 hours, at which time the embolic material was still present in the embolized kidney. Histologic examination of the specimen showed extensive necrosis in the embolized kidney.

### EXAMPLE 32

Embolization of the Internal Carotid Artery in the Rat

In an anaesthetized rat, the internal carotid artery was surgically isolated. 50 microliters of an agent as prepared in Example 15 was injected into the internal carotid artery. Functional CT prior to embolization showed equal perfusion bilaterally. When repeated after embolization, a large fraction of the cortex receiving blood from the isolated internal carotid artery showed nearly absent perfusion but the contralateral cerebral cortex was unaffected. Volumetric CT scanning localized the embolized brain substance ipsilaterally using transverse, coronal and saggital views.

### EXAMPLE 33

Trapping Ability of the Capillary Embolic Agent I

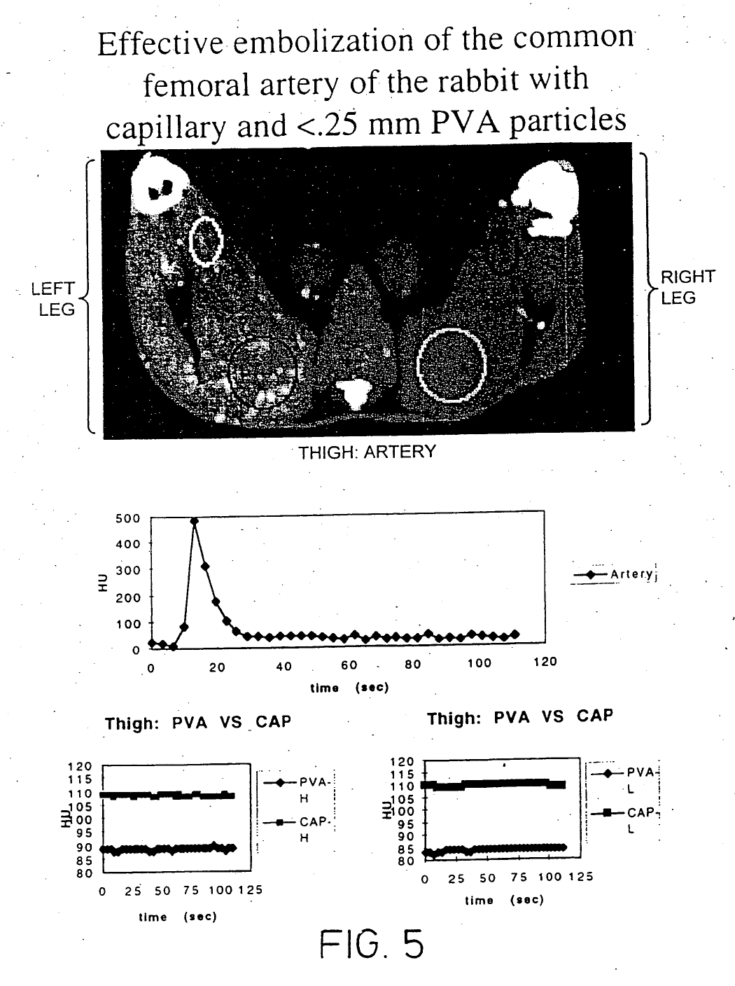

To demonstrate the trapping efficiency of capillary embolic formulation versus conventional PVA embolic particles, a formulation as prepared in Example 14 and a conventional suspension of PVA (150 to 250 microns) were supplemented with iohexol. In a rabbit the right femoral artery was embolized with 4 ml (2 ml of PVA suspension+2 ml of Omnipaque 350) while the left femoral artery was embolized with 2 ml of the agent from Example 14. After embolization, CT scanning of the leg showed little muscular tissue enhancement of the right leg but >60 HU enhancement of the tissues of the left leg. Then, a bolus of omnipaque 350 was administered into the lower abdominal aorta and functional CT performed. This revealed little perfusion of either leg, although the bolus reached the femoral artery on each side. In this case, the water soluble iohexol in the initial capillary embolic agent (i.e., Example 14) formulation reflected the pharmacokinetics of free material that gets trapped in the distal circulation with capillary embolization of the combined material. However, the proximal occlusion caused by the PVA prevents trapping of the coadministered ioxhexol. This is an example of “chemoembolization” with capillary embolic formulations wherein the “chemol” is represented by the added Omnipaque 350. See FIG. 5 of the accompanying drawings.

### EXAMPLE 34

Embolization of the Myocardium

In separate rabbits, the anterior free wall of the myocardium was selectively embolized using a preparation as made in Example 15 from the anterior coronary while the septum was selectively embolized from selective catheterization of the posterior coronary circulation. Further experiments selectively embolized the hepatic and mesenteric arterial circulation using the same formulation.

### EXAMPLE 35

Trapping Ability of the Capillary Embolic Agent II

To demonstrate the trapping efficiency of a capillary embolic formulation versus conventional PVA embolic particles, a formulation was prepared as in Example 15. A conventional suspension of PVA (150 to 250 microns) was supplemented with an insoluble CT X-ray contrast agent as disclosed in Example 19 of WO-A-96/23524 such that the PVA demonstrated a significant amount of X-ray opacity. In a rabbit the right renal artery was embolized with 2 ml of the agent from the PVA suspension while the left renal artery was embolized with 2 ml of the embolic agent from Example 15. Digital subtraction angiography carried out with images of the kidneys before and after the placement of the emboli revealed that the right renal artery was filled with X-ray dense material without opacification within the cortex or medulla of the kidney itself. The left renal artery and the arterial bed of the left kidney were filled with X-ray dense material suggesting embolization of the entire organ. After embolization, CT scanning of the kidney showed little enhancement of the right kidney but excellent enhancement of the right renal artery. The left kidney was completely filled with X-ray dense material from the capillary embolic agent.

Thus, the capillary embolic agent was efficient at embolizing the target tissue and retaining the soluble agent within the embolus after placement of the embolus.

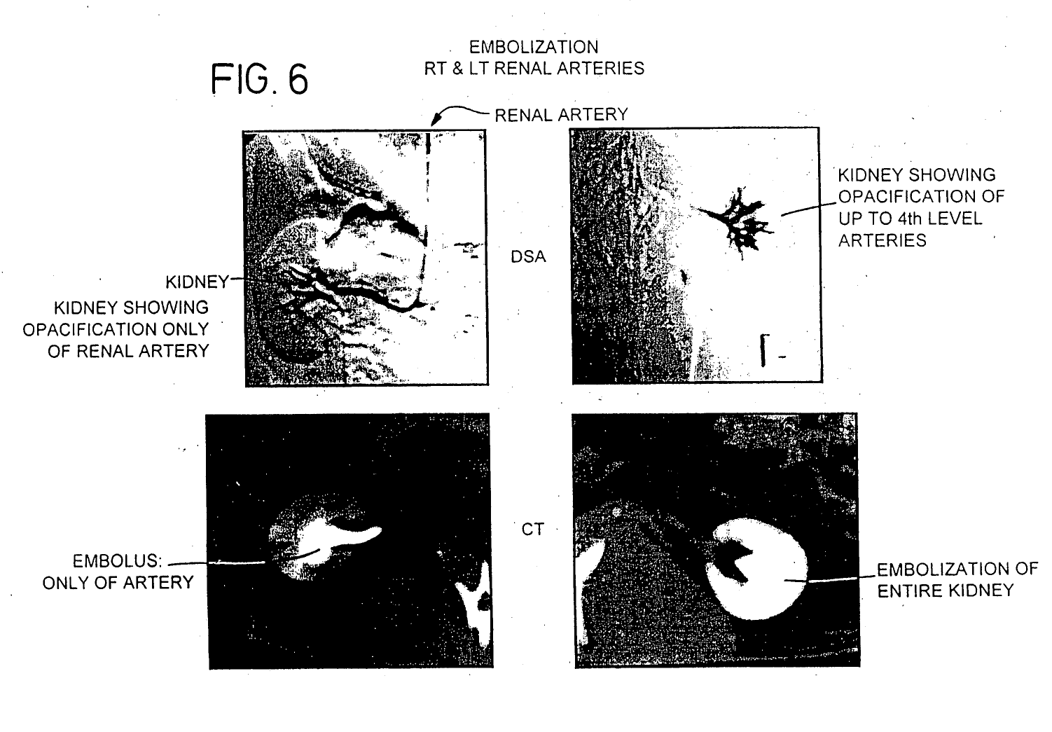

See FIG. 6 of the accompanying drawings.

### EXAMPLE 36

Trapping Ability of the Capillary Embolic Agent III

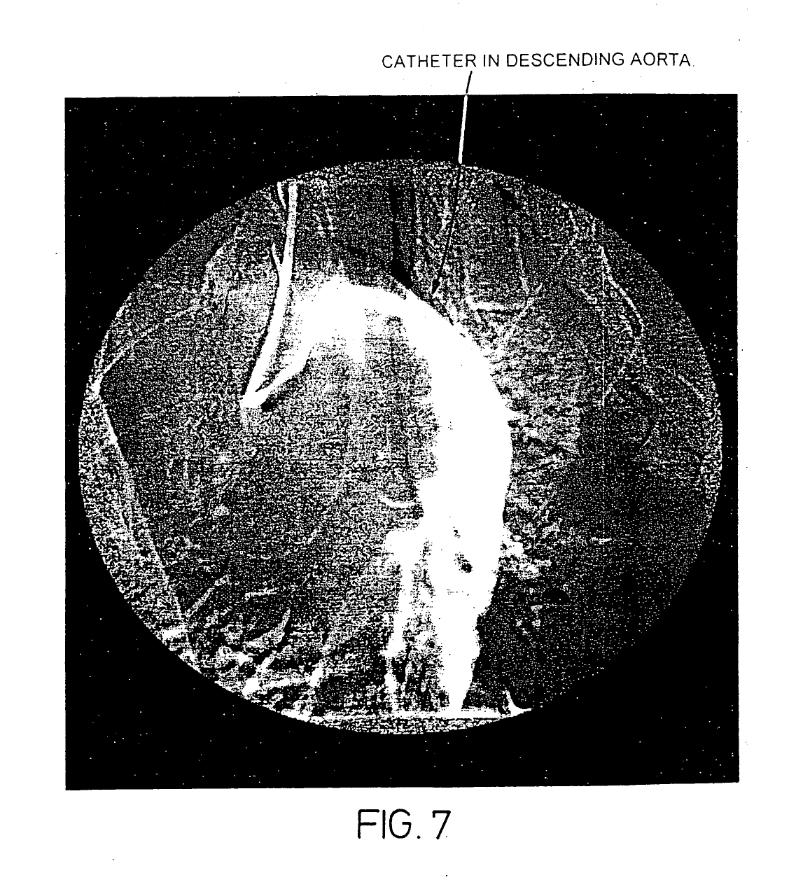

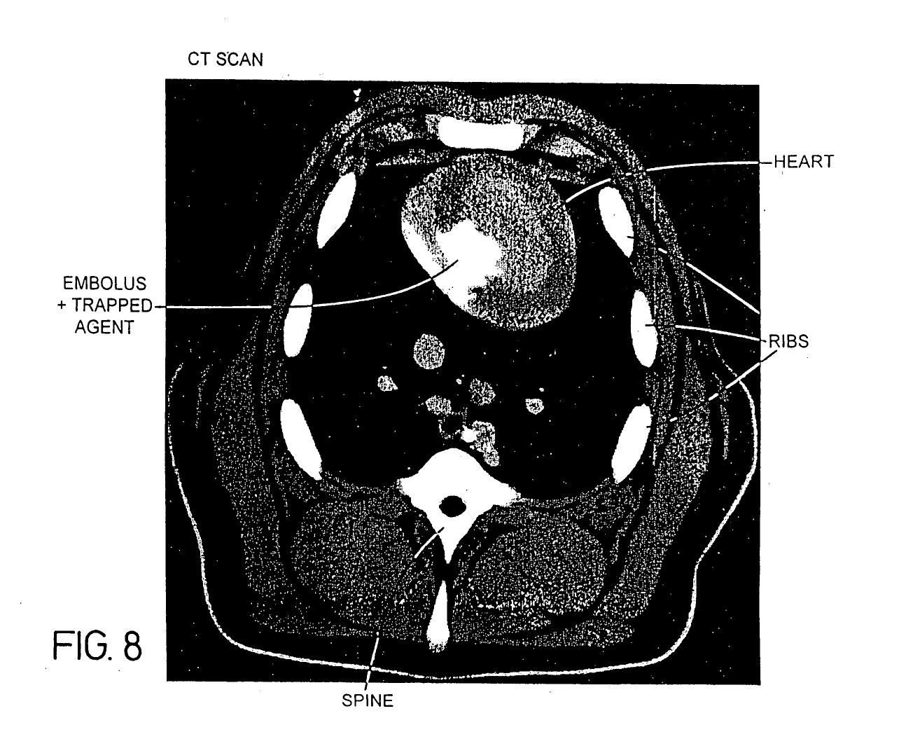

A branch of the coronary artery of an anaesthetized pig was catheterized and the posterior circulation embolized with an embolic agent as prepared in Example 14. The placement of the catheter is shown by digital subtraction angiography (see FIG. 7 of the accompanying drawings). A subsequent CT scan (see FIG. 8 of the accompanying drawings) taken at about the mid-left ventricle showed retention of the trapped water soluble radio-opaque agent in the posterior papillary muscle and adjacent septum, as well as in the right ventricular free wall.

1-7. (canceled)

8. A method of radiation therapy of a tissue comprising the steps of:
i) administering into the vasculature including the capillaries of a perfused zone of tissue in a human or non-human animal subject a composition comprising particles of a size or formulation selected to generate emboli at a target site within said subject, characterised in that as said particles are used solid water-insoluble particles 1-50 micrometers in size of a non-radioactive diagnostically effective compound in a non-polymeric particulate matrix selected from the group consisting of insoluble metal oxides, insoluble metal salts, inert metals, glass, ceramic particles and porous particles or vesicles encapsulating a non-radioactive diagnostically effective compound or a solution thereof; and
ii) applying a therapeutic dose of radiation, wherein said particles act as a radiation therapy sensitizer.

9. A method as claimed in claim 8, wherein said composition further comprises a conventional contrast agent.

10. A method as claimed in claim 9, wherein said conventional contrast agent acts as a radiation therapy sensitizer.

11. A method as claimed in claim 8, wherein said therapeutic dose of radiation originates from a source external to said tissue.

12. A method as claimed in claim 8, wherein said therapeutic dose of radiation originates from a source internal to said tissue.

13. A method as claimed in claim 12, wherein said internal source of radiation comprises implanted ¹²⁵I.

14. A method as claimed in claim 9, wherein said conventional contrast agent is a radio-dense material.

15. A method as claimed in claim 14, wherein said radio-dense material comprises an iodinated contrast agent.

16. A method as claimed in claim 15, wherein said iodinated contrast agent is selected from the group consisting of 6-(ethoxycarbonyl)hexyl bis(3,5-acetylamino)-2,4,6-triiodobenzoate (NC67722), (ethoxycarbonyl)methyl bis(3,5-acetylamino)-2,4,6-triiodobenzoate (NC12901), 1-(ethoxycarbonyl)pentyl bis(3,5-acetylamino)-2,4,6-triiodobenzoate (NC70146) and ethyl bis(3,5-acetylamino)-2,4,6-triiodobenzoate (NC8883).

17. A method as claimed in claim 9, wherein said conventional contrast agent is both an MR active and X-ray absorbing material.

18. A method as claimed in claim 17, wherein said conventional contrast agent is selected from the group consisting of gadolinium oxide, gadolinium oxalate and manganese-doped hydroxyapatite.

19. A method of chemoembolic therapy comprising administering into the vasculature of a perfused zone of tissue in a human or non-human animal subject particles of a size or formulation selected to generate emboli at a target site within said subject, in combination with a therapeutic agent, characterised in that as said particles are used solid water-insoluble particles of a non-radioactive diagnostically effective compound or vesicles encapsulating a non-radioactive diagnostically effective compound or a solution thereof, and wherein said therapeutic agent is a promoter of vascular growth.

20. A method of chemoembolic therapy comprising administering into the vasculature including the capillaries of a perfused zone of tissue in a human or non-human animal subject particles of a size or formulation selected to generate emboli at a target site within said subject, in combination with a therapeutic agent, characterised in that as said particles are used solid water-insoluble particles 1-50 micrometers in size of a non-radioactive diagnostically effective compound in a non-polymeric particulate matrix selected from the group consisting of insoluble metal oxides, insoluble metal salts, inert metals, glass, ceramic particles and porous particles or vesicles encapsulating a non-radioactive diagnostically effective compound or a solution thereof, and wherein said therapeutic agent is an inhibitor of vascular growth.

21. A method as claimed in claim 19, wherein said promoter of vascular growth is selected from the list comprising vascular endothelial growth factor (VEGF), vascular endothelial growth-related protein, basic fibroblast growth factors (bFGF and FGF-3), epidermal growth factor, hepatocycte growth factor, insulin-like growth factor, placental growth factor, placental proliferin-related protein, platelet-derived growth factor, platelet-derived endothelial growth factor, proliferin, proliferin-related protein, transforming growth factors α and β and tumor growth factor α.

22. A method as claimed in claim 20, wherein said inhibitor of vascular growth is selected from the list comprising tecogalan sodium (Daiichi), AGM-1470 (Takeda/Abbott), CM101 (Carbomed), mitaflaxone (Lipha), GM-1603 (Glycomed), rPF4 (Repligen), MPF-4 (Lilly), recombinant angiostatin (Entremed), endostatin, thalidomide (Entremed), DC101 (ImClone Systems), OLX-514 (Aronex), raloxifene hydrochloride (Lilly), suramin sodium (Parke-Davis), IL-12 (Roche), marimastat (British Biotech), and CAI (NCI).

23. A method as claimed in claim 19, wherein said therapeutic agent is a cytotoxin.

24. A method as claimed in claim 23, wherein said cytotoxin is selected from the group comprising carboplatin, mitoxantrone, epirubicin, mitomycin C, decarbazine, vinblastine, cisplastin, interferon, dactinomycin, hydroxyurea, carmustine, methyl CNNU, interleukin-2, cyclophosphamide, amsacrine and doxorubicin.

25. A method as claimed in claim 19, wherein said therapeutic agent is a biotherapeutic agent.

26. A method as claimed in claim 25, wherein said biotherapeutic agent is selected from the group consisting of antisense nucleic acids, diphtheria toxin and ricin A chain.

27. A method as claimed in claim 19, wherein said therapeutic agent is a nuclear agent.

28. A method as claimed in claim 19, wherein said particles are administered prior to administration of said therapeutic agent.

29. A method as claimed in claim 19, wherein said particles are administered after administration of said therapeutic agent.

30. A method as claimed in claim 19, wherein said particles are coadministered with said therapeutic agent.

31. A method as claimed in claim 28, wherein said generated emboli are temporary and said therapeutic agent is targeted.

32. A method as claimed in claim 28, wherein said generated emboli are temporary and said therapeutic agent comprises genetic material.

33. A method as claimed in claim 19, wherein said therapeutic agent is a material that enhances another therapeutic intervention.

34. A method as claimed in claim 33, wherein said therapeutic intervention is hyperthermia or photolytic therapy.

35. A method of identifying local pharmacokinetics in tissue comprising administering into the vasculature including the capillaries of a perfused zone of tissue in a human or non-human animal subject particles of a size or formulation selected to generate emboli at a target site within said subject optionally in combination with an imageable agent, characterised in that as said particles are used solid water-insoluble particles 1-50 micrometers in size of a non-radioactive diagnostically effective compound in a non-polymeric particulate matrix selected from the group consisting of insoluble metal oxides, insoluble metal salts, inert metals, glass, ceramic particles and porous particles or vesicles encapsulating a non-radioactive diagnostically effective compound or a solution thereof.

36. Use of solid water-insoluble particles of a non-radioactive diagnostically effective compound or vesicles encapsulating a non-radioactive diagnostically effective compound or a solution thereof as defined in claim 1 for the manufacture of an embolus generating pharmaceutical composition for use in embolus therapy.

37. A pharmaceutical composition comprising embolus forming contrast-effective particles together with a physiologically tolerable sterile liquid carrier medium, characterised in that as said particles are used solid water-insoluble particles of a non-radioactive diagnostically effective compound or vesicles encapsulating a non-radioactive diagnostically effective compound or a solution thereof, as defined in claim 8.

38. A method as claimed in claim 22, wherein said therapeutic agent is an inhibitor of vascular growth.

39. A method as claimed in claim 20, wherein said therapeutic agent is a cytotoxin.

40. A method as claimed in claim 20, wherein said therapeutic agent is a nuclear agent.

41. A method as claimed in claim 20, wherein said particles are administered prior to administration of said therapeutic agent.

42. A method as claimed in claim 20, wherein said particles are administered after administration of said therapeutic agent.

43. A method as claimed in claim 20, wherein said particles are coadministered with said therapeutic agent.

44. A method as claimed in claim 20, wherein said therapeutic agent is a material that enhances another therapeutic intervention.
